(19) United States
(12) Patent Application Publication
(10) Pub. No.: US 2024/0210391 A1
(43) Pub. Date: Jun. 27, 2024

(54) DEVICE TO DETECT AIRBORNE PATHOGENS

(71) Applicant: Jung-Tang Huang, Taipei (TW)

(72) Inventor: Jung-Tang Huang, Taipei (TW)

(21) Appl. No.: 18/369,839

(22) Filed: Sep. 19, 2023

(30) Foreign Application Priority Data
Sep. 20, 2022 (TW) 111135539

(51) Int. Cl. G01N 33/543; A61B 5/00; A61B 5/08; A61B 10/00; G01N 33/53; G01N 33/569

(52) U.S. Cl. CPC G01N 33/54386; A61B 5/082; A61B 5/6803; A61B 10/0051; G01N 33/5308; G01N 33/54346; G01N 33/5695; G01N 33/56983; A61B 2010/0006; A61B 2010/0087; G01N 2333/11; G01N 2333/165; G01N 2333/35

(57) ABSTRACT

A device for detecting airborne pathogens, comprising a mask and a detection test strip; the test strip at least comprising a sample collection area, and a test/control color developing area, the test strip is arranged on the inner side of the mask, aligns the sample collection area with the user's mouth; the structural design of the mask provides a rapid collection unit to collect the droplets generated by the user's speech, singing, coughing, sneezing, or exhalation, into a test sample. After a period of wearing time, when the accumulated droplet volume is enough to pass through the liquid buffer segment, the target pathogens or biochemical molecules bonded with aptamer-modified gold nanoparticles in the flow can arrive test/control color development area; when the user takes off the mask, he can directly observe whether the test/control color development area of the test strip shows a positive reaction with the naked eye.

### BACKGROUND OF THE INVENTION

### 1. Field of the Invention

The present invention relates to a device capable of detecting airborne pathogens, particularly a test piece for detecting airborne pathogens is set on the inner surface of the mask, which may be used to detect whether the wearer of the mask is a spreader of the airborne pathogens.

### 2. Description of Related Art

For the infection of the COVID-19 virus, the current antibody or antigen rapid screening needs to collect respiratory specimens, and the subject is prone to discomfort, and PCR detection is time-consuming and needs to be operated by professional personnel and equipment, etc. In addition, conventional rapid screening reagents, from the nasal cavity or throat or saliva, may have too many false negatives due to incorrect sampling points and non-continuous testing, which has become a breach in epidemic prevention. Furthermore, as to whether the subject is contagious, it is usually necessary to have symptoms, or to be confirmed by PCR testing, but asymptomatic, or pre-symptomatic, contagious patients, close to 59% of the cases (Reference: JAMA Network Open. 2021; 4(1):e2035057. doi:10.1001/jamanetworkopen.2020.35057), and so far it is impossible to prevent.

In addition, due to the pathogens of some airborne diseases, its sample sampling is not easy, such as Mycobacterium tuberculosis, until today, sputum smear microscopy is still the most widely used diagnostic test in resource-limited areas, although its sensitivity is not ideal.

Tuberculosis (TB) is a highly contagious disease associated with significant morbidity and mortality worldwide, with an estimated 10 million new TB cases and over 1.5 million deaths in 2020. Since the tuberculosis is the most contagious disease, and early detection of cases and timely treatment are effective means of interrupting the spread of the disease in the community. Despite limitations, direct smear microscopy, and to some extent culture, form the diagnostic backbone of national tuberculosis programs in high disease burden countries such as India. Direct smear microscopy is a simple and rapid method of detection, but it suffers from a low sensitivity of between 20% and 60%. The culture method is highly sensitive and widely accepted as the “gold standard”; however, it is unacceptably slow and requires anywhere from 2 weeks for liquid culture to 8 weeks for solid culture to provide results. In addition, it is more expensive, more technically demanding, and requires the establishment of a biosafety laboratory.

Alternative diagnostic methods such as nucleic acid amplification tests (NAATs) are of legitimate importance in the diagnosis of tuberculosis by reducing turnaround time and maintaining acceptable sensitivity and specificity. For example, GeneXpert MTB/RIF (Xpert®), line probe assay (LPA) and loop-mediated isothermal amplification (TB-LAMP), which detect tuberculosis and rifampicin (RIF) resistance, have been approved by WHO for test for tuberculosis. However, despite their satisfactory performance in diagnosing tuberculosis, these tests suffer from one or more limitations, including an inability to distinguish between live and dead bacteria, high cost, and the need for a trained workforce, maintenance, and infrastructure, all of which is preventing them from scaling up in resource-constrained settings and in underdeveloped and geographically remote areas. Additionally, reliance on proprietary equipment and reagents, batch-to-batch variability in kit performance, periodic requirements for instrument calibration, and high module replacement rates in Xpert® remain causes of concern, requiring close monitoring of failure rates as key Quality Index. A blood test (LIOSpot TB) that may distinguish between active and latent TB infection in adults was recently reported. However, the test requires a cell culture laboratory setup for isolating peripheral blood mononuclear cells from patients and assessing interleukin 2 (IL-2) production in response to tuberculosis antigens. Therefore, a low-cost, rapid, sensitive, economical, easy-to-use, and accurate high-throughput tuberculosis detection method remains an unmet need. Such testing would facilitate active case detection in high-risk populations and community screening in high disease burden settings, which would help reduce disease transmission and ultimately contribute to TB control in communities.

Several reports describe the utility of detecting Mycobacterium tuberculosis (Mtb) antigens in sputum for the diagnosis of pulmonary tuberculosis. However, the execution efficiency of these tests varies. Antibody detection of Mycobacterium tuberculosis lipoarabinomannose (Mtb LAM) and purified protein derivative (PPD) antigens has a sensitivity of 86% to 95% and a specificity of 15-100%. And a major challenge in implementing antigen-based TB testing is ensuring the availability of scalable high-quality reagents to meet the needs of disease detection and population screening. Although antibodies have long been used as diagnostic reagents, they have several limitations, including lot-to-lot variability, requirements for animal housing and cell culture facilities, and cost. Furthermore, cold chain requirements and the limited shelf life of antibodies are important obstacles to their widespread use.

Therefore, for areas where the COVID-19 virus is prevalent or Mycobacterium tuberculosis is prevalent, or areas where airborne pathogens are prevalent, use necessary epidemic prevention measures and everyone wears a mask. If the masks themselves may continuously collect the droplets of infected people for a long time and have built-in detection functions, the infected people may be screened out early, the chance of false negatives will be reduced, and asymptomatic infected people will have nowhere to hide. To date, no masks are available that may detect bioaerosols exhaled from the mouth and nose of a sick person in real time. The present invention provides a sensing mask to monitor whether the wearer of the sensing mask is infected and has the ability to infect. In other words, if the airborne pathogenic mask of the present invention is aimed at detecting viruses/bacteria, it may directly use the method of wearing a mask to detect viruses/bacteria in the exhaled droplets, which may reduce the discomfort of the subjects, shorten the detection time, cost, and improve detection efficiency and the use of aptamers to directly detect live pathogens such as live viruses/bacteria. If the infection is confirmed, it means that it has the ability to infect and needs to be effectively isolated.

### SUMMARY OF THE INVENTION

One of the purposes of the present invention is to provide a mask that may detect airborne pathogens, especially to detect whether the droplets exhaled by the wearer's mouth and nose have infectious live pathogens.

The second object of the present invention is to provide a mask that may detect airborne pathogens, especially for continuous measurement, to produce a peak viral load in the first three days before asymptomatic or symptomatic infections such as the COVID-19 virus in period of high infectivity.

The third object of the present invention is to provide an innovative design of a mouth mask, which may quickly collect the droplets generated by the user's speech and exhalation, make it gather into a drop of specimen with a volume of at least 10 uL, and concentrate on the sample collection area of the test piece.

The fourth object of the present invention is to provide a device capable of detecting airborne pathogens. The device is a screening test piece, and the test piece includes a carrier substrate on which at least one sample is arranged in sequence. The sample collection area is pre-soaked or dripped into the extraction solution and dried; a liquid buffer material fragment; a conjugate pad is provided with at least one aptamer/gold nanoparticle specific to the target pathogen; a test/control color detection area; wherein the liquid buffer segment allows the sample to continuously concentrate on the liquid buffer segment, when the cumulative sample volume is sufficient to pass through the liquid buffer segment, the pathogen and the aptamer/nano-gold in the flow sample may reach the test/control chromogenic detection area.

The fifth object of the present invention is to provide a device capable of detecting airborne pathogens. The device is a screening test piece integrated into a mask. The test piece includes a carrier substrate, on which at least a sample collection area is set up, pre-soaked or dripped into the extract, and wait for drying; a liquid buffer segment; a conjugate pad, equipped with at least one aptamer/gold nanoparticle specific to the target pathogen; a test/control chromogenic detection area; between the sample collection area and the conjugate pad, a liquid buffer segment may be added to allow the droplets to continuously concentrate on the liquid buffer segment. When the cumulative droplet volume is not enough to pass through the liquid buffer segment, the spit may be used to directly flow pathogens and aptamer gold nanoparticles to the test/control chromogenic detection area in the sample collection area.

The sixth object of the present invention is to provide a device capable of detecting airborne pathogens. The device is a screening test piece, and the test piece includes a carrier substrate on which at least one sample is arranged in sequence. The sample collection area is pre-soaked or dripped with the lysate or extract, and dried; a liquid buffer segment; a conjugate pad, provided with at least one aptamer gold nanoparticles specific to the target pathogen; a test/control chromogenic detection area; add a dry lysing agent or extraction compound to the sample collection area. When in use, allow the dry extract components of the sample collection layer to have enough time to dissolve in the collected droplets and saliva, and at the same time let the extract the target pathogen protein from the pathogen in the droplets and saliva, which may be used to measure whether the target pathogen exceeds the confirmed concentration.

The seventh object of the present invention is to provide a mask that may detect exhaled breath condensate (EBC) and exhaled breath aerosol (EBA). The exhaled breath condensate may include semi-volatile and non-volatile organic compounds, cytokines, proteins, cell debris, DNA and viruses, bacteria. Exhaled aerosol (EBA) fraction in addition to the expected gas and water vapour, exhaled air contains minute aerosols (both liquid and solid particles) generated by surface membrane disruption and upper airway turbulence at the alveolar level.

The eighth object of the present invention is to provide a device capable of detecting airborne pathogens, comprising a mask, an airborne pathogen detection test strip, the test strip at least includes a sample collection area, and a test/control color-developing area, the test piece is set on the inner surface of the mask so that the sample collection area is aligned with the mouth area; the structural design of the mask provides a facility to quickly collect the droplets produced by the user's speech and exhalation, so that they may be gathered into a droplet with a volume of 20-80 uL of sample, and concentrated in the sample collection area of the test strip. After wearing for a period of time, the user may take it off and visually observe whether the test/control color-developing area of the test strip presents a positive or negative reaction.

The ninth object of the present invention is to provide a mask that may detect airborne pathogens. Users do not need to use a sampling stick to rub the nasal cavity, then insert a buffer, and then drip into the rapid test piece. Instead, the user only needs to put on the mask of the present invention, and after a period of time, take it off to see if the test/control color area of the test piece shows a positive or negative reaction. If the control line does not turn red, it means that the sampling is still insufficient. He may continue to wear the mask until the control line turns red. This means that the sampling is correct, and thus the positive or negative reaction may be accurately judged.

The tenth object of the present invention is to provide a device that may detect airborne pathogens. The device may be a screening test piece. The user does not need to use a sampling stick to rub the nasal cavity, and mix with the buffer, and then drop into the quick screening test strip. The user only needs to spit directly into the sample collection area until the control line turns red, which means that the sampling is correct, and thus the positive or negative reaction may be accurately judged.

The eleventh object of the present invention is to provide a device that may detect airborne pathogens. The device may be a screening test piece combined with a mask. The user does not need to use a sampling stick to rub the nasal cavity, and mix with the buffer, and then drop into the quick screening test strip. The user only needs to put on the mask of the present invention, and after a period of time, such as from morning to night, take it off to see the test of the test piece whether the test/control color-developing shows a positive or negative reaction. If the control line does not turn red, it means that the amount of sampled solution is still insufficient. One may spit directly into the sample collection area until the control line turns red. This means that the sampling is correct, and thus the positive or negative reaction may be accurately judged.

The twelfth object of the present invention is to provide a mask that may detect airborne pathogens, allowing users to wear a mask for a long time, and the screening test piece may be used for a long time, as long as the control line does not appear red, this test strips are still effective. Compared with the traditional quick screening test strips, they need to be used within half an hour after unpacking, otherwise they will be invalid. For pathogens lurking in the throat or lungs, using droplets produced by speaking, singing, coughing, sneezing, or exhaling may have a better chance of early detection, that is, pathogens in the upper or lower respiratory tract, or even the lungs may be sampled non-invasively and naturally.

The thirteenth object of the present invention is to provide a non-lysis screening test piece that may be installed in a general medical mask. By wearing it for a day, the virus may be accumulated and attached to the sampling area of the screening test piece. Finally, the wearer may spit saliva to the test piece for self-monitoring. The structure of the non-lysis test strip is similar to that of the commercially available quick screening test piece. The difference is that the lysate is added to the sample collection area in advance, and a hydrogel block is added as the liquid buffer segment. This method may improve the specimen binding efficiency and detection effect.

The fourteenth object of the present invention is to provide a mask that may detect airborne pathogens. The detection method is not affected by the variant of the COVID-19, and directly utilizes the corresponding enzyme of the COVID-19 to invade the host organ tissue receptor, such as ACE2 (angiotensin-converting enzyme 2, angiotensin-converting enzyme 2), which is invaded by COVID-19 in human lung cells, uses electrochemical methods to detect the concentration of COVD-19 virus.

Based on the above, the present invention provides a mask that may detect exhaled condensate (EBC) and exhaled aerosol (EBA). The exhaled breath condensate may include semi-volatile and non-volatile organic compounds, cytokines, proteins, cell debris, DNA and viruses, bacteria. Exhaled aerosol (EBA) fraction in addition to the expected gas and water vapour, exhaled air contains minute aerosols (both liquid and solid particles) generated by surface membrane disruption and upper airway turbulence at the alveolar level.

### BRIEF DESCRIPTION OF THE DRAWINGS

In order to fully understand the purpose, characteristics and effects of the present invention, the present invention is described in detail below by following specific embodiments, and in conjunction with the accompanying drawings.

FIG. 1A is a schematic diagram of a hydrogel block disposed at the end of the sample collection area of the test strip according to a preferred embodiment of the present invention.

FIG. 1B is a preferred implementation of the present invention; after a period of wearing time, when the accumulated droplet volume makes the hydrogel block expand enough to connect the conjugate pad.

FIG. 2A is a schematic diagram of the detailed structure of the detection test piece and the mask that may detect airborne pathogens in a preferred embodiment of the present invention, using a detection aptamer and an aptamer complementary DNA.

FIG. 2B is a schematic diagram of a combination of a test strip and a mask capable of detecting airborne pathogens according to a preferred embodiment of the present invention.

FIG. 3A is a schematic diagram of the detailed structure of the detection test strip and the mask for detecting airborne pathogens according to another embodiment of the present invention, the droplet collection stage.

FIG. 3B is a schematic diagram of the detailed structure of the detection test piece and the mask that may detect airborne pathogens according to another embodiment of the present invention, and the stage where the target pathogens in the droplets are combined with aptamer gold nanoparticles.

FIG. 3C is a schematic diagram of the detailed structure of the detection test piece and the mask that may detect airborne pathogens according to another embodiment of the present invention, the target pathogen is combined with the aptamer nano-gold conjugate and the aptamer of the test line, and the aptamer of the control line then combine with non-target gold nanoparticles.

FIG. 3D is a schematic diagram showing the color development of test lines and control lines on a test strip capable of detecting airborne pathogens according to another embodiment of the present invention.

FIG. 4A is a schematic diagram of the detailed structure of the detection test strip and the mask for detecting airborne pathogens according to another embodiment of the present invention, at the droplet collection stage.

FIG. 4B is a schematic diagram of the detailed structure of the detection test strip and the mask that may detect airborne pathogens according to another embodiment of the present invention, and there is no target pathogen in the droplets.

FIG. 4C is a schematic diagram of the detailed structure of the detection test strip and the mask that may detect airborne pathogens according to another embodiment of the present invention. The aptamer of the test line has no target binding, and the aptamer of the control line is bound to non-target gold nanoparticles.

FIG. 4D is a schematic diagram of a control line color displayed on a test strip capable of detecting airborne pathogens according to another embodiment of the present invention.

FIG. 5A is a schematic diagram of a detection test strip capable of detecting airborne pathogens according to another embodiment of the present invention. A single detection aptamer is used, and there is no target pathogen in the droplets.

FIG. 5B is a schematic diagram of a detection test strip capable of detecting airborne pathogens according to another embodiment of the present invention. A single detection aptamer is used, and the target pathogen is contained in the droplets.

FIG. 6 is a schematic diagram of a test strip capable of detecting airborne pathogens according to another embodiment of the present invention, using ACE2 enzyme.

FIG. 7 is a schematic diagram of a combination of a detection test piece and a mask capable of detecting airborne pathogens according to another embodiment of the present invention.

FIG. 8A is a photo of a mask capable of detecting airborne pathogens and FIG. 8B is the mask simulated wearing with a model human head.

FIG. 9A is a schematic diagram of the results of direct saliva sampling for subject-A.

FIG. 9B is a schematic diagram of test results of subject-A after wearing for 6 hours.

FIG. 9C is a schematic diagram of the test results of subject-A after wearing for 12 hours.

FIG. 10A is a schematic diagram of the results of direct saliva sampling for subject-B.

FIG. 10B Sis a schematic diagram of the test results of subject-B after wearing for 6 hours.

FIG. 10C is a schematic diagram of the test results of subject-B after wearing for 12 hours.

### DESCRIPTION OF THE EMBODIMENTS

Breathing is a rich medium that includes gas-phase inorganic and organic compounds, as well as exhaled breath condensate (EBC) and exhaled breath aerosol (EBA). Vapor-phase organic compounds include environmental exposures to volatile organic compounds (VOCs) as well as endogenous metabolites for health diagnostic applications. Exhaled breath condensate may distinguish most non-polar VOCs, including semi-volatile and non-volatile organic compounds, cytokines, proteins, cellular debris, DNA and viruses, bacteria. Exhaled aerosol (EBA) fraction in addition to the expected gas and water vapour, exhaled air contains minute aerosols (both liquid and solid particles) generated by surface membrane disruption and upper airway turbulence at the alveolar level. These aerosols mobilize material that would otherwise be part of the liquid layer of the lungs, so they are part of the exhaled breath condensate.

Saliva is a complex biological mixture that may consist of salivary gland secretions, gingival crevicular fluid, sputum, and/or mucous membrane exudates, the proportions of which depend on the method of collection. Some studies tested only oral secretions, others explicitly tested “retro-oropharyngeal” or “deep throat” saliva with oropharyngeal secretions. Generally speaking, if the droplets produced by talking or singing are mostly saliva, but the droplets produced by coughing or sneezing mostly contain more secretions from the nasal cavity and throat, so using a mask to collect droplets is mainly diversity, it may even collect secretions or biochemical molecules from the gas emitted from the stomach and viscera.

Since the mask collects droplets from breathing, speaking or singing, coughing or sneezing for a long time, it has a comprehensive collection effect, especially singing or speaking, which needs to use the vibration of the vocal cords of the throat, so the mucous of the throat surface, secretions or infections may all become part of the exhaled droplets. As far as sampling is concerned, it may be obtained naturally without invading the throat, and it may be worn for a long time, such as 10 hours, if one breathes 12 times per minute, equivalent to 7,200 times exhales be collected, mixed with droplets produced by speaking or singing, coughing or sneezing.

In terms of the detection of infectious pathogens, using aptamers to directly detect pathogens, if the sensitivity is sufficient, it may also be quantified, especially if the time for collecting the specimen is very short, a positive reaction occurs, basically, it may be inferred that the user is highly contagious and belongs to a super spreader.

According to literature, [Xiaojian Xie, Yuguo Li, Hequan Sun and Li Liu, “Exhaled droplets due to talking and coughing” J. R. Soc. Interface (2009) 6, S703-S714], the general people are singing or speaking or when coughing, the total mass of droplets produced is as follows: The total mass of droplets collected using a surgical mask and a plastic bag with an inner tissue is shown in Table 1. Considerable subject variability was observed, consistent with variability in droplet size distribution measurements. An average of 22.9 mg of fluid was obtained during 20 coughs using the surgical mask method and 85 mg using the plastic bag with tissues. During the count to 100, an average of 18.7 and 79.4 mg of fluid were measured using the mask and plastic bag, respectively.

TABLE 4
Total mass of droplets collected using surgical
face mask and plastic bag with tissue inside.

| | weight (mg) | | | | | | | | | average |
|---|---|---|---|---|---|---|---|---|---|---|
| activity | M1 | M2 | M3 | M4 | M5 | M6 | M7 | F1 | F2 | (mg) |

talking (counting from 1 to 100)

| | | | | | | | | | | |
|---|---|---|---|---|---|---|---|---|---|---|
| mask | [symbol] | 41.8 | 61.3 | — | — | — | — | 15.7 | 1 | 18.7 |
| | 5.5 | | | | | | | 12.6 | 7.7 | |
| bag | 69.4 | 48.8 | 121.7 | — | — | — | — | 113.7 | 66.2 | 79.1 |
| | [symbol] | | | | | | | 151.5 | | |

coughing ( [symbol] 0 times)

| | | | | | | | | | | |
|---|---|---|---|---|---|---|---|---|---|---|
| mask | 44.9 | 30.4 | 17.5 | — | — | — | — | 15.1 | [symbol] | 22.9 |
| | | | | | | | | [symbol] | | |
| | | | | | | | | 16.2 | | |
| bag | 154.5 | [symbol] | [symbol] | 62.7 | 67.4 | 41.8 | [symbol] | 41.4 | 55.4 | [symbol] |

J. R. Soc. Interface (2009) [symbol]
indicates data missing or illegible when filed

It may be seen that the present invention properly designs the surgical mask or the N95 mask, which may effectively collect at least 20 uL of specimen in a short time when singing or speaking or coughing.

In addition, the common rapid screening test piece needs about 20 uL of the sample to mix with about 80 uL of buffer solution or reaction solution, so the present invention provides a kind of mask that may detect airborne pathogens, including a mask, an airborne pathogen screening test piece, the test piece at least includes a sample collection area, and a test/control color development area, the test piece is arranged on the inner surface of the mask, so that the sample collection area is aligned with the mouth area; the structural design of the mask provides a sample collection area that may quickly collect the droplets generated by the user's speech and exhalation, gather them into a sample drop with a volume of 20 uL, and concentrate on the test piece. After wearing for a period of time, the user may take it off and visually observe whether the test/control color-developing area of the test strip presents a positive or negative reaction. If the control line does not turn red, it means that the amount of sampled solution is still insufficient. The user may spit directly into the collection area until the control line turns red. This means that the sampling is correct, and thus the positive or negative reaction may be accurately judged.

If the traditional COVID-19 rapid screening test piece is directly fixed on the mask, its disadvantages are: (1), it may not be used for a long time, because the antibodies on the rapid screening test piece may not be exposed to the general environment for a long time and are easily damaged. (2), it is unacceptable to provide specimens in a distributed manner, because the mask may only collect a small amount of intermittent spit produced by speaking or singing, which may result in insufficient one-time large amount of coloring on the test line and control line. (3), it is impossible to directly import the exhaled sample into the sample collection area. The user needs to use a sampling stick to collect the sample from the nostril, etc., and then insert the sampling stick into the extract in the test tube to extract the N protein or S protein or RNA of the virus. Wait, stir for about one minute, and finally drop the mixture into the sample collection area of the test piece.

The mask of the present invention that may detect pathogens in the exhaled medium needs to be able to overcome the above-mentioned shortcomings. If the embodiment uses the COVID-19 as the detection target, 1) the aptamer of the N protein of the COVID-19 may be used to detect whether the subject's exhaled bioaerosol contains the COVID-19, which is different from the antibody since the aptamer has long-term stability; 2) Just fix the lysis solution that may extract the N protein on the sample collection layer of the test strip and let the lysis solution dry; 3) Use a liquid valve or a liquid buffer segment to be placed behind the sample collection layer, before the conjugate layer or the conjugate pad, so that the dry lysis components of the sample collection layer have enough time to dissolve in the collected droplets, and at the same time the N protein of the COVID-19 in the droplets can be extracted, the liquid valve may be opened to allow the extracted sample to flow through the conjugate layer, and the lateral flow test piece is used to measure whether the N protein of the COVID-19 exceeds the confirmed concentration. The method is not subject to variation of the viral S protein.

In some embodiments, the aptamer selected by the present invention may also be aimed at the S protein of the COVID-19, so that the aptamer has versatility and may not be mutated by the S protein of the virus, which obviously helps to directly convert the captured virus on the sensing chip, without the use of extraction liquid, and the complete virus is directly detected on the screening test piece. In some embodiments, the extraction liquid that will not destroy the virus S protein may also be used to improve the sensitivity of detection.

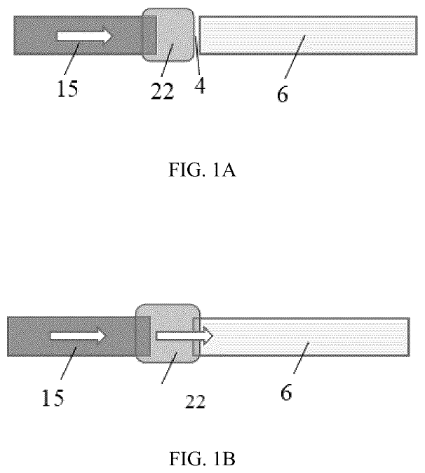

The embodiment of the above-mentioned liquid valve, as shown in FIGS. 1A and 1B, the liquid valve is arranged between the sample collection layer 15 and the conjugate pad/detection zone 6 by using hydrogel or called a liquid buffer segment 22, the liquid buffer segment 22 maintains a small distance 4 from the conjugate pad/detection zone 6. The liquid buffer segment 22 keeps the droplets concentrated, for example, about 60 uL is accumulated in the sample collection layer 15, which is enough for the hydrogel to expand enough to connect the conjugate pad/detection zone 6. Once connected, the flow rate may be greatly increased, and the test line and control line may be colored. The use of hydrogel to absorb water expansion is enough to allow the sample to be collected enough to flow through it at one time. At the same time, the dry extract components of the sample collection layer have enough time to dissolve in the collected exhaled droplets, and at the same time, the pathogens in the collected exhaled droplets are extracted to their specific proteins, so the detection sensitivity may be increased.

The present invention has multiple methods for making screening test pieces, and the steps are as follows:

### Method 1: Sandwich Aptamer-Lateral Flow Test Piece (Sandwich Apt-LFA) Using Single Aptamer and Aptamer Complementary DNA

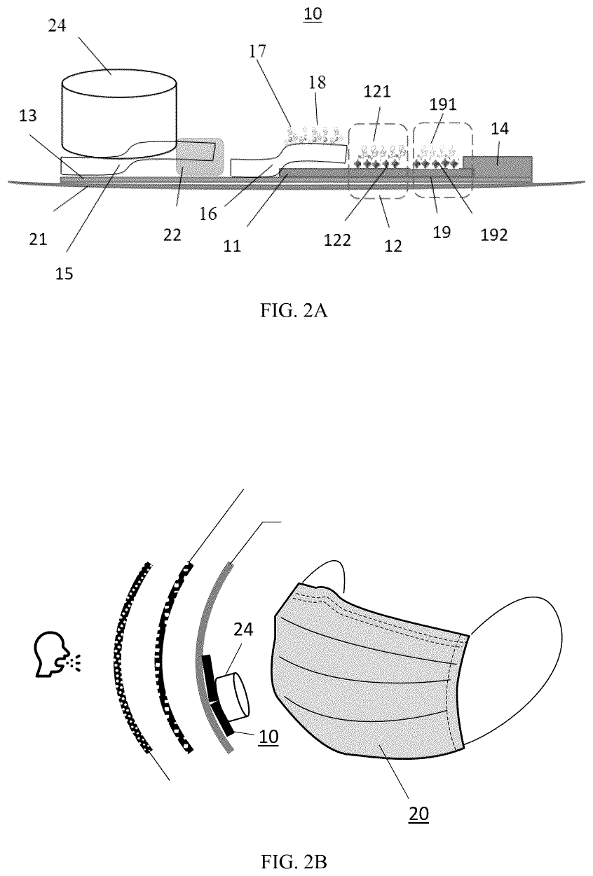

With reference to FIG. 2A and FIG. 2B, the manufacture method of screening test piece 10, on carrier plate 13
Step 1: first fix streptavidin 122 on the test line 12 and streptavidin 192 on the control line 19 on the nitrocellulose membrane substrate 11.
Step 2: combine the biotinylated aptamer 121 specific to the target pathogen with the streptavidin 122 on the test line 12; simultaneously, the complementary DNA segment 191 of the aptamer for control is combined with the streptavidin 192 on the control line 19.
Step 3, the right end of the nitrocellulose membrane substrate 11 is provided with an absorbing layer 14, which is used to guide the sample to effectively flow through the test line 12 and the control line 19.
Step 4: sample collection layer 15, conjugate pad 16 fixes two aptamer-modified gold nanoparticles (AuNPs) thereon, and the aptamer combines with AuNPs through thiolation, one of which is a control aptamer/AuNPs 17 are used, and the other is aptamer/AuNPs 18 for detection. As shown in FIGS. 2A and 2B, a liquid buffer segment 22 is placed between the sample collection layer 15 and the conjugate pad 16, thus completing the test strip 10. The liquid buffer segment 22 allows the droplets to continuously concentrate on the liquid buffer segment 22, and when the accumulated droplet volume is sufficient to pass through the liquid buffer segment 22, the pathogens and aptamer/AuNPs may flow to the detection area.
Step 5: the screening test piece 10 is attached to the inner layer of the mask 21.
Step 6: Cover another droplet collection funnel or C-shaped mouthpiece 24, aim at the mouth, so that the droplets of the user's mouth may be effectively concentrated in the sample collection area or the sample collection layer 15, and the making of mask 20 for the virus detection is completed.

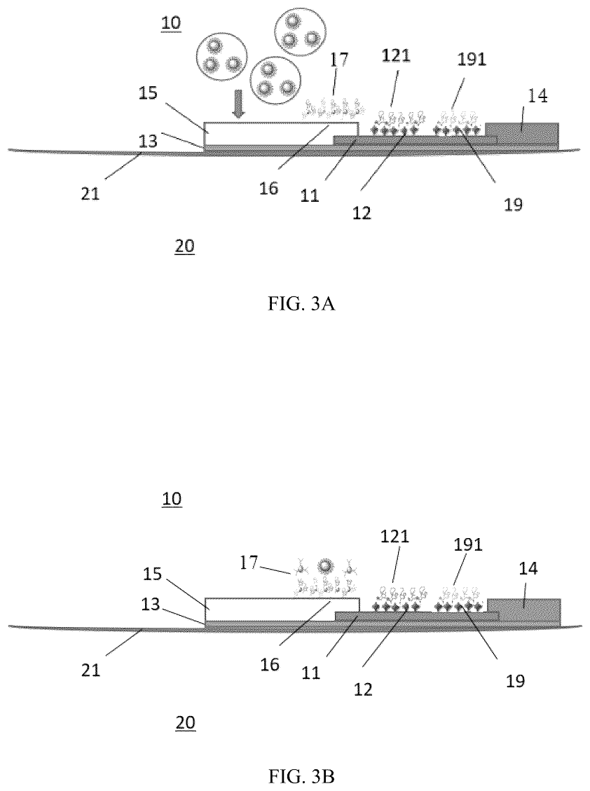

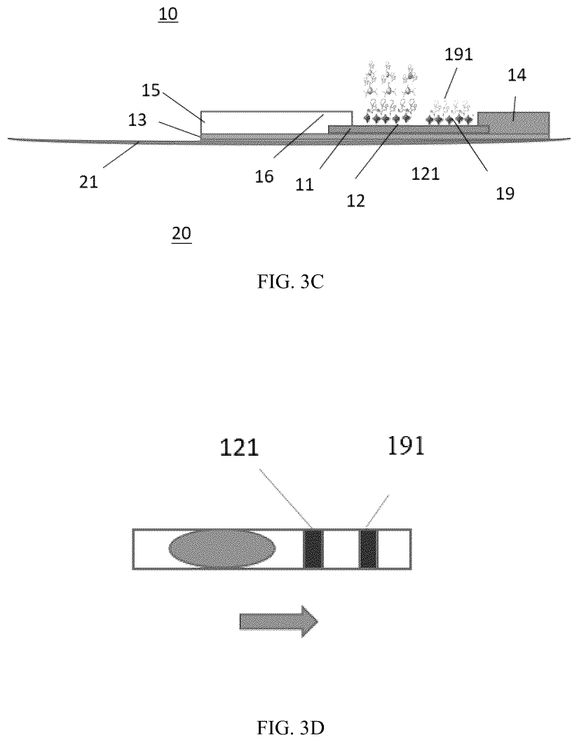

In some embodiments, with reference to FIGS. 3A and 3B, the conjugate pad 16 uses hydrogel, which may make its water content high, such as using hydroxyethyl methacrylate (HEMA), and increase its porous structure, especially the pore size is about 300-1000 nanometers for virus detection, and two aptamer-modified AuNPs may be embedded in these pores.

When the user breathes, especially when speaking or singing or sneezing, the droplets (or bioaerosol) are released through the mouth and attached to the conjugate pad 16. The pathogens in these bioaerosols, such as viruses or germs, will diffuse and combine with the aptamer/AuNPs 18 for detection in the conjugate pad 16. When the amount of water accumulated by the droplets exceeds the water saturation of the hydrogel, pathogens are mixed with the aptamer/AuNPs 18 for detection, and the aptamer/AuNPs 17 for control. The mixed solution will be due to the formation of the target analyte/AuNP-coupled detection aptamer complex after the sample containing the target analyte is loaded and migrated to the conjugate pad 16 by capillary action. The target analyte then continues to migrate along the strip to the test area, where the complex is captured by the capture aptamer 121 and leads to the aggregation of AuNPs (shown in red, FIG. 3D). Excess complexes then pass through the test line 121 and are captured by immobilized oligonucleotide sequences complementary to specific regions of the detection aptamer on the control line 191, resulting in another red band. In samples without the target analyte, the distinct red band is only shown at the control line 191 (FIG. 3D). The user may take off the mask and observe whether the control line 191 and the test line 121 are red.

In some embodiments, the conjugate pad 16 uses hydrogel, and at the junction of the conjugate pad 16 and the nitrocellulose membrane substrate 11, a liquid buffer segment is provided before use, especially during storage and transportation. The moisture in the hydrogel is blocked from being attracted by the capillary force of the nitrocellulose membrane substrate 11, which causes the aptamer gold nanoparticles in the conjugate pad 16 to be dragged, resulting in failure and malfunction of the test piece. The embodiment of this liquid buffer may be that gelatin may absorb water and may be dissolved. In the present invention, before use, the water content in the hydrogel is not enough to release the gelatin. After use, because the exhaled droplets are continuously absorbed by the hydrogel, which causes the water content in the hydrogel to be supersaturated, so the water is released to dissolve the hydrogel, and the buffer opens.

For applications that require a longer period of time to collect the amount of droplets, such as Mycobacterium tuberculosis, the initial water content of the hydrogel may be lower, or the volume of the gelatin valve may be larger, so that the droplets must accumulate a sufficient amount for dissolving gelatin, the amount of water in the sample collection area may be collected for several hours to ensure that the target analyte may be effectively collected.

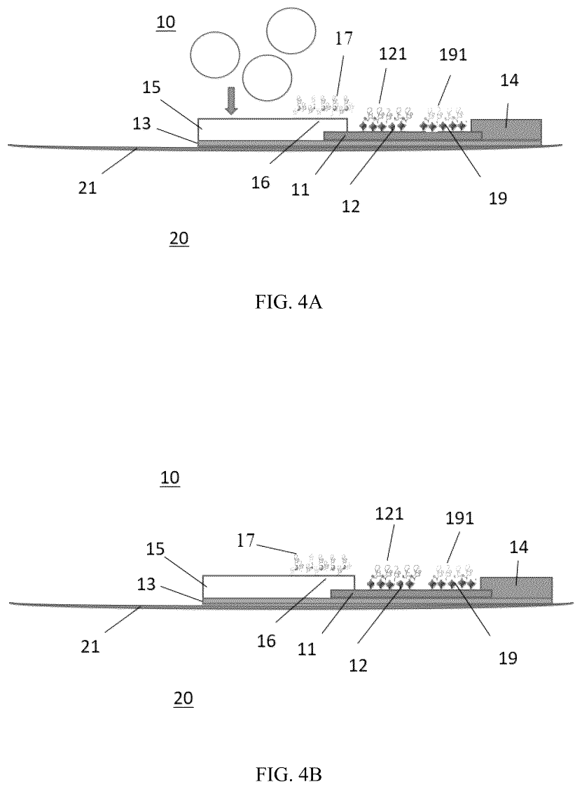

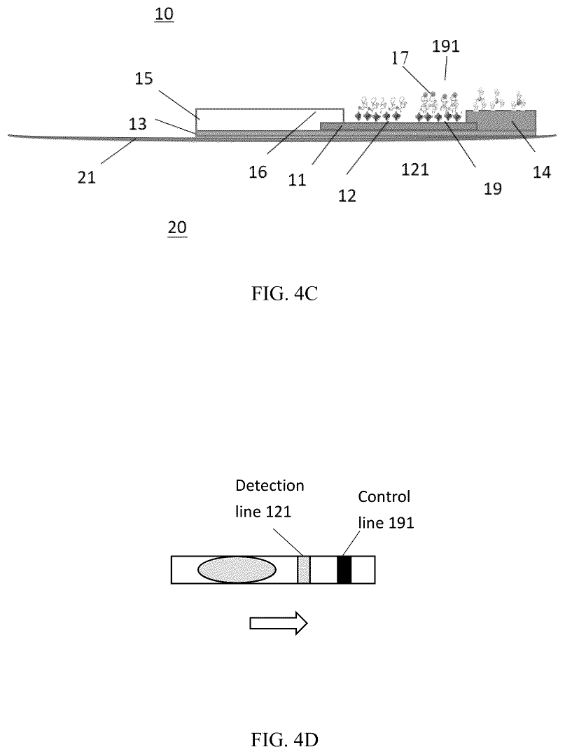

With reference to FIGS. 3A-3D and FIGS. 4A-4D, using the method of virus detection mask 20 and screening test piece 10, when the user puts on the virus detection mask 20 of the present invention, because of the innovative design of the mask, after a period of time, the droplets produced by the user's singing, sneezing, speaking and exhalation may be effectively collected, gathered into a sample drop with a volume of 10 uL, and concentrated in the sample collection area of the test strip 10. After the sample containing the target analyte is loaded and migrates to the conjugation pad 16 by capillary action, a target analyte/AuNP-coupled detection aptamer complex is formed. The target analyte then continues to migrate along the strip to the test zone, where the complex is captured by the capture aptamer and leads to aggregation of AuNPs (shown in characteristic red, FIG. 3D). Excess complexes then pass through the test line and are captured by an immobilized oligonucleotide sequence that is complementary to a specific region of the detection aptamer on the control line, resulting in another red band. In samples without the target analyte, the bright red band is only shown in the control line (FIG. 4D). The user may take off the mask and observe whether the control line and test line are red.

In some embodiments, the user may take off the mask, drip saliva into the sample collection area, and then wait for 15 minutes to observe whether the control line and the test line are red, which is mainly to avoid the collection of droplets not enough to go through the liquid buffer segment 22.

### Method 2: Use a Single Aptamer Sandwich Aptamer-Lateral Flow Test Piece

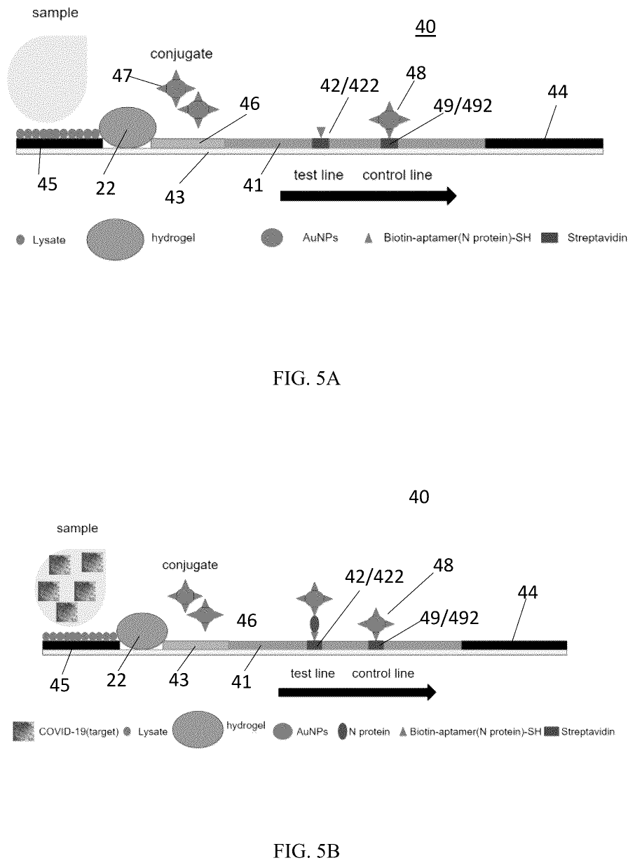

In this embodiment, the N protein of the COVID-19 is still used as the detection target. Referring to FIG. 5A and FIG. 5B
Step 1: first fix streptavidin/biotinylated aptamer 422 on test line 42 on nitrocellulose membrane substrate 41, and streptavidin 492 on control line 49.
Step 2, the right end of the nitrocellulose membrane substrate 41 is provided with an absorbing layer 44, which is used to guide the sample to effectively flow through the test line 42 and the control line 49.
Step 3: The sample collection layer 45 is pre-soaked or dripped into the extract lysate, and it is dried, and a biotinylated aptamer modified gold nanoparticles (AuNPs) 48 is fixed on the conjugate pad 46, the aptamer is highly specific to the target N protein, and the aptamer binds to gold nanoparticles (AuNPs) through thiolation. A hydrogel block or a liquid buffer segment 22 is placed between the sample collection layer 45 and the conjugate pad 46, thus completing the test piece 40. The liquid buffer segment allows the droplets to continuously concentrate on the liquid buffer segment, and when the accumulated droplet volume is sufficient to pass through the liquid buffer segment, the pathogens and aptamer gold nanoparticles may flow to the detection area.
Step 4: With reference to FIG. 2B, the screening test piece 40 is attached to the inner layer of the mask 21.
Step 5: Referring to FIG. 2B, cover a droplet collection funnel 24 and align it with the mouth, so that the droplets from the user's mouth may be effectively concentrated in the sample collection area, and the production of the virus detection mask 20 is completed.

When this virus detection mask 20 is in use, refer to FIG. 5A. When the sample does not have target pathogens, the biotinylated aptamer-modified gold nanoparticles 48 on the conjugate pad 46 will be brought to the control line 49 and binds to the streptavidin 492, so the control line will appear red, and the result is called a negative reaction.

Please refer to FIG. 5B. When the sample has a target pathogen, the target pathogen will be lysed by the extract before the sample collection layer 45, which will produce the required target N protein and some biological substances on the conjugate pad 46. The gold nanoparticle 48 modified by the primed aptamer will be brought to the test line 42 through the combination of the target N protein and streptavidin/biotinylated aptamer 422, therefore the test line will appear red. At the same time, the partially biotinylated aptamer-modified gold nanoparticles 48 will be brought to the control line 49 to combine with streptavidin 492, so the control line will appear red, and the result is called a positive reaction.

### Method 3: Sandwich Aptamer-Lateral Flow Test Piece Using Double Aptamer

Referring to FIGS. 3A-D and 4A-D, a pair of aptamers at different sites of the target analyte is used. First, one of the aptamers, the detection aptamer, was bound to gold nanoparticles (AuNPs) by thiolation and loaded onto the conjugate pad as a recognition element. The second aptamer was biotinylated and immobilized on the test line by streptavidin-biotin conjugation (streptavidin was precoated on nitrocellulose membrane) as a capture aptamer. After the sample containing the target analyte was loaded and migrated to the conjugate pad by capillary action, the target analyte/AuNP-coupled detection aptamer complex was formed. The target analyte then continues to migrate along the strip to the test zone, where the complex is captured by the capture aptamer and leads to aggregation of AuNPs (shown in red, FIG. 3D). Excess complexes then pass through the test line and are captured by an immobilized oligonucleotide sequence that is complementary to a specific region of the detection aptamer on the control line, resulting in another red band. In samples in the absence of target analyte, the bright red band is only shown in the control line (FIG. 4D). The above-mentioned target analytes generally refer to proteins, viruses, and bacteria related to airborne pathogens. In fact, by immobilizing two different aptamers on the conjugate pad and the test line to recognize different sites of the target analyte, many assays showed high specificity and sensitivity in both target spiked buffers and clinical samples. In some embodiments, the same aptamer may also be selected for the aptamer on the conjugate pad and the test line. If there are many aptamer binding sites for the target analyte, it may still exhibit high specificity and sensitivity, but it may reduce the cost of aptamers.

### Method 4: Sandwich Aptamer-Lateral Flow Test Piece Using Split Aptamer

The mechanism of split aptamer design is based on target-induced recombination of aptamer fragments. In the presence of the target molecule, two separate aptamer fragments may regain the three-dimensional structure and restore the affinity of the parental aptamer. By conjugating one fragment of the aptamer to a signal reporter (e.g. gold nanoparticles), and immobilizing the other fragment on the test region (used as a capture agent), a sandwich LFA may be created.

In conclusion, although combined aptamer/antibody and split aptamer strategies have been developed in recent years, double aptamer-based sandwich LFAs are the first choice for highly sensitive and specific LFA development. Further advances in aptamer identification techniques, including the use of efficient and diverse initial libraries (e.g., G-quadruplex libraries), next-generation sequencing (NGS)-based candidate identification, and rational counter selection strategies (e.g., using aptamer binding sites inhibitors) will help facilitate the development of double-aptamer-based high-quality LFAs.

### Method 5: Using the Aptamer-Lateral Flow Test Piece of ACE2 Enzyme

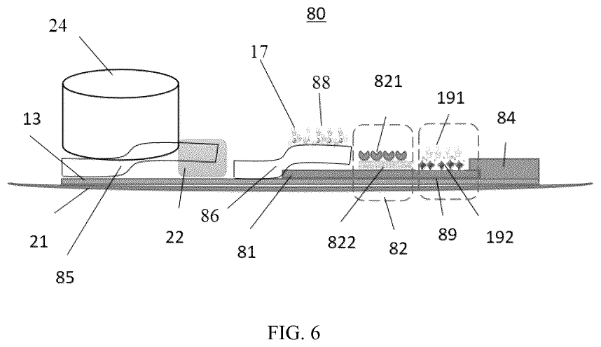

Referring to FIG. 6, the present invention proposes early detection of SARS-CoV-2 using the ACE2 enzyme as a recognition element to achieve clinically relevant detection. The test provides a scalable route to sensitive, specific, rapid and low-cost large-scale testing.

SARS-CoV-2 has four major structural proteins, and the spike protein is known to bind to the surface of cells expressing angiotensin-converting enzyme 2 (ACE2) on their surface. The affinity between ACE2 and the spike protein has been shown to be in the low nM range, resulting in a similar affinity level for antibody-antigen interactions. Due to the high affinity between the spike protein and ACE2 and the fact that a limited number of coronaviruses utilize ACE2 for entry (SARS-CoV-1, SARS-CoV-2, and HCoV), this enzyme represents an important candidate molecule for constructing biosensors. Thus, ACE2 has the potential to be deployed as a selective receptor in various biosensor formats for this key class of human respiratory pathogens for definitive diagnosis of SARS-CoV-2 in adults, or as a screen tool to identify positive cases, then undergo lab testing for confirmation.

The sensor of the present invention requires a simple two-stage fabrication procedure: (1) depositing 1H,1H,2H,2H-perfluorododemayethiol (PFDT) 822 on the test line and (2) ACE2 821 adsorbed into PFDT 822 is functionalized (FIG. 6). (3) Furthermore, the assay is not affected by viral mutations as it exploits the interaction between the SARS-CoV-2 Spike protein and ACE2, which has a somewhat built-in surface orientation through the ability of ACE2 to intercalate through its hydrophobic region, and provides an opportunity to develop similar tests for cell entry by other respiratory viruses through membrane-bound surface proteins.

With reference to FIG. 6, the manufacture method of screening test piece 80, on carrier plate 13,
Step 1: first immobilize streptavidin 192 on the nitrocellulose membrane substrate 81 at the control line 89.
Step 2: ACE2 821 specific to the target pathogen is combined with the PFDT 822 on the test line 82; simultaneously, the complementary DNA fragment 191 of the aptamer is combined with the streptavidin 192 on the control line 89.
Step 3, an absorbing layer 84 is provided on the right end of the nitrocellulose membrane substrate 81 to guide the sample to effectively flow through the test line 82 and the control line 89.
Step 4: Sample collection layer 85, conjugate pad 86 fixes two aptamer-modified gold nanoparticles (AuNPs) thereon, and the aptamer combines with gold nanoparticles (AuNPs) through thiolation, one of which is a control aptamer gold nanoparticle 87 was used, and the other was the aptamer gold nanometer 88 used to detect the spike protein. As shown in FIG. 6, a liquid buffer segment 22 is placed between the sample collection layer 85 and the conjugate pad 86, thus completing the screening test piece 80. The liquid buffer segment 22 allows the droplets to continuously concentrate on the liquid buffer segment 22, and when the accumulated droplet volume is sufficient to pass through the liquid buffer segment 22, the pathogen and spike protein aptamer gold nanoparticles may flow to the detection area.

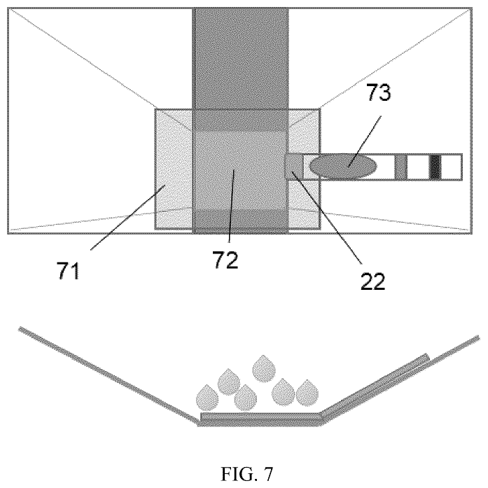

With reference to FIG. 7, it is another embodiment of the present invention, the surroundings of the pathogen screening test piece of the mask use hydrophobic non-woven material 71, when people wear the mask of the present invention, if they are completely close to each other, as long as they speak, just may produce a lot of mist, these mist may form water droplet on the surface of hydrophobic non-woven fabric, and may gather more and more and become big water droplet, just like saliva, may roll into pathogen screening test piece specimen collection area 72 at last, therefore sample may be collected. The collection area 72 is located in the center of the mouth area. The nose breathing area uses standard non-woven fabric, which is breathable. In addition, the liquid buffer segment 22 and the conjugate pad 73 of the pathogen screening test piece, the test line and the control line, etc. may also be covered with hydrophobic materials to avoid the adhesion of the droplets and cause interference.

In some embodiments, in order to improve the collection efficiency, the mask may be an N95 mask, and N95 has a filtration efficiency of >95% and a bacteria filtration efficiency of >99% for suspended submicron particles. The edge of the N95 respiratory protective mask is well sealed, and the shape of the mask conforms to the contour of the human face, which may prevent suspended submicron particles from entering or flowing out of the gap between the edge of the mask and the surface of the face.

### Embodiment One: To Detect Mycobacterium tuberculosis

The screening test piece provided inside the mouth mask according to the embodiment of the present invention may be based on various airborne pathogens, including influenza, Ebola virus, Zika virus, or organophosphorus nerve agents, and other airborne pathogens, such as Mycobacterium tuberculosis (M. tb), etc., are specifically configured with corresponding aptamers. In the case of Mycobacterium tuberculosis, aptamers against various M. tb have been developed in the literature, and feasible TB targets include Mycobacterium tuberculosis virulence factors (FbpA, FbpB and Fpb), Mycobacterium tuberculosis-specific proteins (phosphate-binding transport tuberculosis protein PstS1), M. tuberculosis extracellular antigens (MPT64 and MPT51), endothelial M. tuberculosis-specific proteins (α-Crystalline; Acr and HspX) and soluble M. tuberculosis proteins (CFP-2, −10, −30 and ESAT-6), mannose-capped lipoarabinomannan (ManLAM), etc.

In the embodiment of the present invention, a DNA aptamer-based M. tb diagnostic test is disclosed, and the aptamer of the COVID-19 in FIG. 2 to FIG. 5 is replaced with the aptamer for the M. tb biomarker HspX (SEQ ID NO 1), 5′-GGGAACAATATGTTCAAGGGCTCTTTAAAGTTTTAGTTCGTTTG-3′, or SEQ ID NO 2 5′-AGGGCTTTTTTTTTTTTTAGTTCGTTTG-3′ for the direct detection of the M. tb biomarker HspX in exhaled or spittle or droplets. The mask of the present invention that may detect airborne pathogens is compared with the performance of aptamer-linked immobilized sorbent assay (ALISA) and enzyme-linked immunosorbent assay (antibody ELISA) based on anti-HspX polyclonal antibody. The present invention may be more convenient and efficient, and superior to smear microscopy, antibody-based ELISA and chest X-ray for detecting tuberculosis. This has potential utility for active case-finding in high-risk populations and for tuberculosis screening in subjects with suspected M. tb.

In addition, in order to improve the accuracy, specificity, and sensitivity of detecting M. tb, in some embodiments, an all-in-one M. tb aptamer may be provided, such as an aptamer using MPT64 antigen, and aptamers against lipoglycan on the surface of ManLAM, or aptamers against the whole M. tb pathogen. That is to say, if the number of test lines is increased to two or three, the types of corresponding aptamer gold nanoparticles also increase correspondingly. The main surface lipoglycan of Mycobacterium tuberculosis (M tb), ManLAM is mannose-capped lipoarabinomannan, which is the immunosuppressive epitope of M. tb. ManLAM is a unique surface lipoglycan component or constantly released from Mycobacterium tuberculosis (M tb) cell wall, which makes it a perfect candidate biomarker for TB diagnosis.

The DNA sequence of the MPT64 antigen aptamer is as follows

MPT64 antigen aptamer I (MAA I)
(SEQ ID NO 3):
5′-SH-(CH₂)₆-TGGGAGCTGATGT-
CGCATGGGTTTTGATCACATGA-3′
MPT64 antigen aptamer II (MAA II)
(SEQ ID NO 4):
5′-SH-(CH₂)₆-TTCGGGAATGATTATCAA-
ATTTATGCCCTCTGAT-3′

The aptamer ZXL1 of the ManLAM antigen (SEQ ID NO 5) is as follows

5′-biotin-GGCGCCATAG CGACGGGGCC
ATTCCAAGAA-SH-3′

The aptamer for the whole M. tb germ H37Ra (SEQ ID NO 6) is as follows

5′-biotin-TTGGTTGCTG AATCCCCTCG
TCTTGGCTTC TTTGTCGGG-SH-3′

### Embodiment 2: Simultaneous Detection of H1N1 Influenza and COVID-19

Because H1N1 influenza and COVID-19 are easy to spread at the same time, and their symptoms are very similar, it is not easy to distinguish, but the infectiousness of the two is different, the medicines for treatment are also different, and the way of treatment is different from the degree of fatality. Therefore, it is very important to distinguish at the first time. The screening test piece of the present invention may increase the test line to two or more. If two test lines are taken as an example, and with reference to the design of the lateral flow screening test piece shown in FIG. 5, the first test line detects COVID-19 and the second test line detects H1N1 flu. The corresponding aptamers are:

The sequence of the aptamer used to detect the N protein of COVID-19 is as follows, and the aptamer-adapted biotin is used to immobilize on the streptavidin of the first test line.

biotin-Aptamer-SH (SEQ ID NO 7):
5′-biotin-GCTGGATGTCGCTTACGACAATATT
CCTTAGGGGCACCGCTACATTGACACATCCAGC-3′

The sequence of the aptamer used to detect the HA antigen of influenza H1N1 is as follows, and the biotin adapted to the aptamer is used to immobilize on the streptavidin of the second test line.

biotin-Aptamer-SH (SEQ ID NO 8):
5′-biotin-GTACTTCCGGACCAGTT-GTCTT
TCGGTCTCTACCCCCAGCCCGTCAAAAGTG-SH-3′

In this way, the embodiment of the present invention may be used to simultaneously detect H1N1 influenza and COVID-19 with very similar symptoms. It is worth noting that in the extended embodiment, the Mycobacterium tuberculosis detection in the first embodiment may also be integrated with the COVID-19 and influenza virus in the second embodiment to form a three-in-one pathogen detection mask.

### Embodiment Three

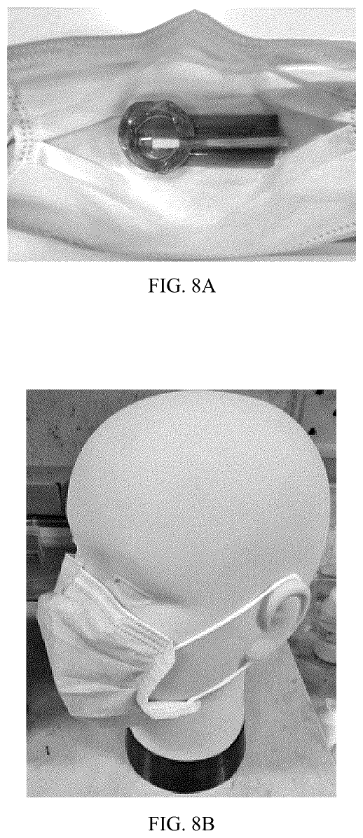

The air-borne pathogen detection mask designed for general wear may not only protect the wearer but also protect others. The purpose is to know whether there is infection after wearing for a period of time, so that the wearer may be able to know whether he is infected at the first time, thus may also greatly reduce the risk of virus spread, and do medical treatment at the first time, reducing the excessive waste of medical treatment. The appearance of the simulated assembly of the test piece is shown in FIGS. 8A and 8B.

The embodiment of the present invention utilizes the collection of the wearer's saliva and exhaled droplets as the sample, and the difference from the commercially available rapid screening test piece is that the binding and sensing regions use nucleic acid aptamers instead of antibodies, and aptamers that match specific viruses connected to the detection area (test line), and using the specificity of the aptamer, the virus and the aptamer are joined to the sensing area of the designed screening test piece, and the color of the labeled particles is used for qualitative analysis.

The current rapid screening test based on antibody needs to swab respiratory specimens from the nose or throat, and the subject is prone to discomfort. Compared with this, the virus detection mask directly uses the general mask wearing to detect the virus in the exhaled droplets, which may reduce the discomfort of the subject. Prolong detection time to improve detection sensitivity and use aptamers to directly detect live viruses. If infection is confirmed, it means that the viruses have the ability to infect.

This design is assembled and integrated on the mask based on the concept of rapid screening reagents. It is different from commercially available rapid screening using antibodies. Instead the present invention employs aptamers to capture target viruses (ex: H1N1, COVID-19, etc.) for detection. After completion of each fragment (sample collection pad, conjugate pad, nitrocellulose membrane) of the rapid screening test piece, it is cut according to the specifications of the commercially available rapid screening test piece for subsequent assembly (simulation, physical assembly). The size of each fragment varies. The length is the same, the sample collection pad and the conjugate pad are both 1.4 cm, the nitrocellulose membrane (NC membrane) is 2.5 cm, and the absorbent pad is 2 cm, the width is 4 mm, and the overlap of each other is 4 mm. The only difference is the liquid buffer segment of hydrogel block placement between the sample collection pad and the conjugate pad. There is no overlap between the two segments and the distance is about 3-3.5 mm. The hydrogel block has the function of time-delay, so that the extract may have sufficient reaction time with the pathogen to facilitate the effect of subsequent detection.

The material of the hydrogel block used in the embodiment of the present invention is sodium polyacrylate (acrylic sodium salt polymer, ASAP) powder, which has the hydrophilic property of hydrogel to absorb water and expand. Use 0.1 g of ASAP powder, and ultrapure water (HPLC) as the liquid source for making hydrogel blocks. The aggregation of the powder is by titrating 3000 μL of ultrapure water. The face of the initial titration is the front side, then the reverse side (that is, the bottom side), each titration is 1000 μL, and a total of 3000 μL of ultra-pure water is dropped on each of the front and back sides. After the titration is completed, stir it fully (tweezers, spoons, and flat clips are all acceptable). After stirring, it is confirmed that the overall hydrogel group is transparent and has no white powder blocks. Then dry it into a hydrogel block with a closed cover at a specific temperature (for example, 40° C.).

First assemble each segment into a regular rapid screening test piece, and embed the assembled test piece into a general surgical mask (or N95 mask). The sensing area of the test piece is used for relevant detection of specific targets (ex: H1N1 HA protein, COVID-19, etc.). The liquid buffer segment (hydrogel block) is covered by an arch-shaped hydrophobic sheet. The arch height is 3 mm, the purpose is to prevent the detection droplets from directly interfering with the hydrogel block when it falls to the sampling area (sample collection pad) and let the hydrogel block after absorbing the liquid expand toward the absorption pad (moving as far as possible to the direction of the absorption pad instead of expanding randomly in all directions).

In the embodiment of the present invention, after the test piece is physically assembled, it is integrated into a general wearable mask with a C-shaped mouthpiece, as shown in FIG. 8A, which is a complete physical assembly and simulated wearing with a model human head, respectively as FIG. 8B.

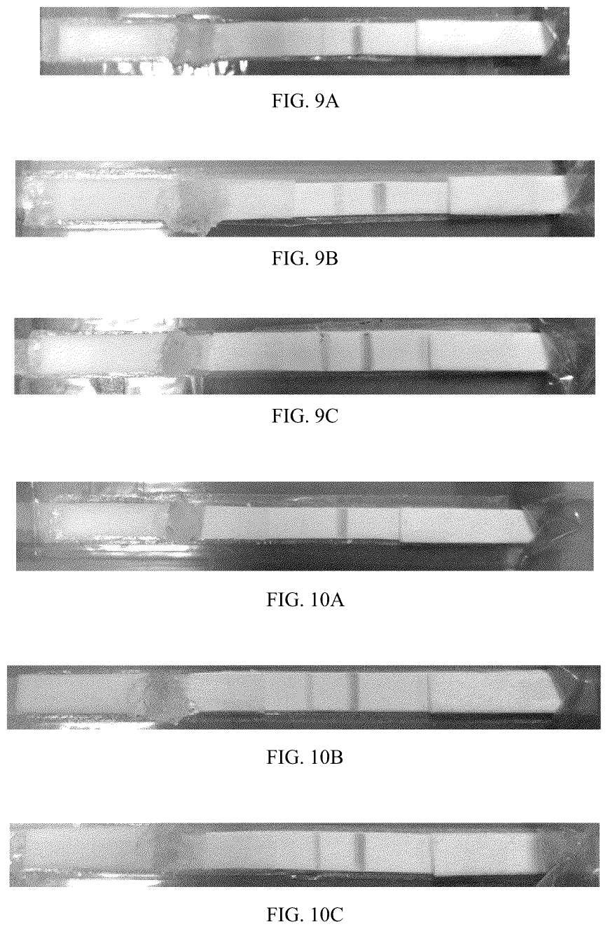

After the virus screening test piece is integrated into the mask, the subject wears it for a long time and observes the result that the virus in the droplets accumulates on the sensing test piece and affects the sensitivity (the lines are more obvious and the color is darker) through wearing for a long time, due to health safety and regulatory considerations, this experiment was outsourced to legal professional institutions and personnel to record the symptoms of the subjects and ask the subjects to help take pictures, record the results of the test and send them back. There are three subjects in total. Due to concerns about personal information, two of them are represented by subjects A and B. The symptoms of subject A are severe cough, sore throat (serious pain or tingling), nasal congestion, and wheezing. The symptoms of subject B are severe cough and sore throat (cutting), nasal congestion, runny nose, and loss of taste. Each subject was self-diagnosed by quick screening and positive PCR test before performing this experiment. Subject A was tested 9 days after the onset of symptoms, and subject B was tested 11 days after the onset of symptoms. In the test, each subject in this experiment wore a total of three designed masks. Each mask was set as the situational conditions of direct saliva collection (spitting), continuous wearing for 6 hours and 12 hours. The experimental results after the arch-shaped hydrophobic layer is removed for easy observation, are shown in FIGS. 9A-9C and FIGS. 10A-10C.

Through the experimental results, it is found that the subjects may be detected by directly sampling saliva (spit) and by wearing for a long time (6-12 hours) to collect and accumulate the virus in the mouth. The virus detection test strip designed in this experiment may be used to detect whether it is infected (positive). Although the detection time has been 9 or 11 days since the onset of symptoms, the virus load is relatively low. Obviously, as the mask continues to be worn for 6 hours, the test line is relatively obvious with saliva sampling (spitting) again, and the test line is more obvious after changing the mask and wearing it for 12 hours. This result shows that the test strip may be used for a long time. Compared with the traditional rapid screening test strip, it needs to be used within half an hour after unpacking, otherwise it will be invalid. For pathogens lurking in the throat or lungs, the droplets produced by speaking, singing, coughing, sneezing, or exhaling may be accumulated to obtain more sensitive test results. Therefore, within three days before the diagnosis, if wearing the mask of the present invention has more opportunities for early detection, that is, non-invasive natural sampling of pathogens in the upper respiratory tract or lower respiratory tract, and even pathogens in the lungs.

Embodiment 3 of the present invention provides a non-lysis pathogen screening test piece that may be installed in a general medical mask. By wearing it for a day, the virus may accumulate and attach to the sample collection area of the test piece. Finally, the wearer may directly use the saliva to drive test strips for self-monitoring. The structure of the lysis-free test piece is similar to that of the commercially available rapid-screening, the difference is that the lysate is added to the sample collection pad in advance, and a hydrogel block is added as a sample buffer material to enhance the binding efficiency between target and the aptamer. In the embodiment of the present invention, the detection of H1N1 HA protein and COVID-19 N protein has been obtained through experiments, and the minimum detection concentration is 0.1 ng/ml. In addition, it has also been verified by actual wearing of COVID-19 patients who have been sick for more than 9-11 days and COVID-19 patients two to three days before the onset. It was found that the present invention may collect and accumulate the target virus to achieve the effect of early detection and prevention.

In summary, the embodiments of the present invention are characterized as follows:

Because of natural sampling, multi-sampling, sampling times up to thousands of times, false negatives are reduced, no need to pick nose, or go deep into the throat, it will not make the subject uncomfortable, and may avoid improper sampling (nostril picking, or deep into the throat) or fear of pain, resulting in false negatives.

For the COVID-19, extract its N protein from the virus in the collected oral droplets, and use the lateral flow test piece (LFA) to measure whether the N protein of the COVID-19 exceeds the confirmed concentration. It is mutated by the virus S protein, that is, it may be used universally for COVID-19 and is not affected by the mutation.

The sensitivity is not lower than that of the usual rapid screening, and the target may reach 0.1 ng/mL. Because the sample collection pad contains the extract, it may effectively obtain the specific protein inside the pathogen and detect it with the aptamer.

Because the aptamer is used as a probe, the user may carry and store the mask of the present invention with himself. If he wears it for three hours, or six hours, or 12 hours, or longer, he may continue to use it, accumulate more specimens, and then spit directly at the sample collection layer to perform the detection.

It is most effective for asymptomatic carriers and pre-symptomatic carriers of the COVID-19 virus, which may greatly reduce the chain of transmission, especially for pathogens with high infection rates. For example, select the subjects who have been in contact with the confirmed person for more than 15 minutes, select the front-line personnel who have served in the medical unit for a long time, select the personnel who need to dispose of medical waste, and select the front-line service personnel of the restaurant.

For infectious diseases that are not easy to rely on a single sampling, such as Mycobacterium tuberculosis, etc., the present invention provides the natural sampling, multi-sampling, and the sampling frequency is as many as thousands of times, plus configures a variety of different aptamers for antigens or proteins or different parts of Mycobacterium tuberculosis, which may greatly increase specificity, sensitivity, and accuracy.

Multiple pathogens may be detected at the same time, such as H1N1 influenza, COVID-19, Mycobacterium tuberculosis, etc. After infection by these three pathogens, the symptoms are similar, and the invention may effectively distinguish the type of pathogens infected.

It is easy to preserve and is not affected by ambient temperature due to the reliability of the aptamer.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.
The name of the XML: facemask.xml
The date of creation: 2024/03/18
The size of the XML file in bytes: 8.02 KB

What is claimed is:

1. A device for detecting airborne pathogens, comprising a mask and a screening test piece for

airborne pathogens, the test piece includes a carrier substrate, on which at least
a sample collection area, which is pre-soaked or dripped with an extract or lysate, and dried into a dry extract compound,
a segment of liquid buffer,
a conjugate pad, provided with at least one kind of aptamer-modified gold nanoparticles specific to the target pathogens,
a test/control color detection zone,
the test piece is set on the inner surface of the mask, the structure of the mask is designed to provide a droplet concentration unit aimed at the user's mouth above the sample collection area to collect the droplets produced by the user when speaking, singing, coughing, sneezing, or exhaling, so that it is concentrated in the sample collection area and the liquid buffer segment of the test piece, after a period of wearing time, when the accumulated droplet volume is sufficient, or when the droplet volume is insufficient, the user spits directly into the sample collection area to let the droplet or saliva to dissolve the dry extraction compound to extract the target analyte of the pathogens in the droplet or saliva, and then pass through the liquid buffer segment, and then the target analyte in the sample is flowed with the aptamer-modified gold nanoparticles on the conjugate pad to the test/control color development detection area, the user directly observes with the naked eye whether the test/control color development detection area of the test strip presents a positive reaction.

2. The device according to claim 1, wherein the droplet concentration unit includes a C-shaped mouthpiece matching the user's mouth and a hydrophobic layer surrounding the sample collection area to increase the capture rate of the sample.

3. The device according to claim 1, wherein the liquid buffer segment is selected from hydrogel, one end of which is connected to the sample collection area, and the other end is kept in a gap with the conjugate pad without contact, after a period of liquid sample collection time , allowing the hydrogel to swell and connect with the conjugate pad, the pathogens or biochemical molecules bonded with the aptamer-modified gold nanoparticles in the flowing droplets reach the test/control color development detection area.

4. The device according to claim 1, wherein the screening test piece production program comprising of, on the carrier substrate
(a) immobilizing streptavidin on the nitrocellulose membrane substrate, on the test line and the control line,
(b) combining the biotinylated aptamer specific to the target pathogen with the streptavidin on the test line, at the same time, combine the complementary DNA fragment of the control aptamer with the streptavidin on the control line,
(c) arranging an absorbing layer at the right end of the nitrocellulose membrane substrate to guide the sample to effectively flow through the test line and the control line,
(d) immobilizing two kinds of aptamer-modified gold nanoparticles on the conjugate pad, and the aptamers combined with gold nanoparticles through thiolation, one of which is the aptamer-modif1ed gold nanoparticles for control, and the other for the aptamer-modified gold nanoparticles for detection, a liquid buffer segment is placed between the sample collection area and the conjugate pad, thus completing the screening test piece.

5. The device according to claim 1, the screening test piece production program comprising of, on the carrier substrate,
(a) immobilizing streptavidin on the nitrocellulose membrane substrate, on the test line and the control line,
(b) binding the aptamer for capture, that is, the biotinylated aptamer specific to the target pathogen, to the streptavidin on the test line,
(c) arranging an absorbing layer at the right end of the nitrocellulose membrane substrate to guide the sample to effectively flow through the test line and the control line,
(d) immobilizing the aptamer for detection on the conjugate pad, two options for the aptamer, one is the same as the aptamer for capture, and the other is different from the aptamer for capture, the aptamer is thiolated with gold nanoparticles (AuNPs) combined into aptamer gold nanoparticles,
(e) combining the complementary DNA fragment of the aptamer for detection with streptavidin on the control line, placing a liquid buffer fragment between the sample collection area and the conjugate pad, thus completing the screening test piece.

6. The deVice according to claim 1, wherein the screening test piece production program comprising of, on the carrier substrate
(a) immobilizing streptavidin/biotinylated aptamer on the test line on the nitrocellulose membrane substrate, and streptavidin on the control line,
(b) arranging an absorbing layer on the right end of the nitrocellulose membrane substrate to guide the sample to effectively flow through the test line and the control line,
(c) pre-soaking or dripping the sample collection layer into the extract, and after it is dried, a biotinylated aptamer modified gold nanoparticles (AuNPs) is immobilized on the conjugate pad, which is compatible with the extracted target analyte, and the highly specific aptamer is combined with gold nanoparticles (AuNPs) through thiolation, a hydrogel block or a liquid buffer segment is placed between the sample collection layer and the conjugate pad, thus completing the screening test piece.

7. The deVice according to claim 1, wherein the screening test piece production program comprising of, on the carrier substrate
(a) immobilizing streptavidin (streptavidin) on the nitrocellulose membrane substrate, and placing it on the control line,
(b) conjugating angiotensin-converting enzyme 2 (ACE2) specific for the target pathogen to 1H, 1H, 2H, 2H-perfluorododemayethiol (PFDT) in the test line, the body binds to streptavidin on the control line,
(c) arranging an absorbing layer at the right end of the nitrocellulose membrane substrate to guide the sample to effectively flow through the test line and the control line,
(d) immobilizing two aptamer-modified gold nanoparticles on the conjugate pad, for the aptamer-modified gold nanoparticles used for detecting the spike protein, a liquid buffer segment is placed between the sample collection layer and the detection area of the conjugate pad, thus completing the screening test piece.

8. The device according to claim 1, wherein the pathogens are selected from exhaled breath condensate (BBC) and exhaled aerosol (EBA), exhaled breath condensate includes semi-volatile, non-volatile, volatile organic compounds, cytokines, proteins, cellular debris, DNA and viruses, bacteria, exhaled aerosols contain microscopic aerosols produced by surface membrane disruption at alveolar level and upper airway turbulence.

9. The device according to claim 1, wherein the pathogens are selected from one or a combination of COVID-19 virus, influenza virus, Mycobacterium tuberculosis, Ebola virus, Zika virus, and norovirus.

10. The device according to claim 1, wherein the aptamers specific to the target analytes of the pathogens are selected from one or a combination of Mycobacterium tuberculosis virulence factors (Fpr, FbpB and pr), Mycobacterium tuberculosis-specific proteins (phosphate-binding transport tuberculosis protein PstS1), M. tuberculosis extracellular antigens (MPT64 and MPT51), endothelial M. tuberculosis-specific proteins (ct-Crystalline, Acr and HspX) and soluble M. tuberculosis proteins (CFP-Z, -10, -30 and ESAT-6), surface lipoglycan (ManLAM).

11. A device for detecting airborne pathogens, comprising a detection test strip, the test strip includes a carrier substrate, on which at least
a sample collection area, pre-soaked or dripped into the extraction solution, and allowed to dry into a dry extraction compound,
a segment of liquid buffer material,
a conjugate pad, provided with at least one kind of aptamer-modif1ed gold nanoparticles specific to the target pathogens,
a test/control color detection zone;
the user directly spits saliva into the sample collection area; so that the saliva dissolves the dry extraction compound to extract the target analyte of the pathogens in the saliva, and then passes through the liquid buffer segment to flow the extracted target analyte in the sample; and binds with the aptamer-modified gold nanoparticles on the conjugate pad; and finally arrives at the test/control color detection area; the user directly observe with the naked eye whether the test/control chromogenic detection area of the test strip presents a positive reaction.

12. The device according to claim 11; wherein the liquid buffer segment is selected from hydrogel; one end of which is connected to the sample collection area; and the other end is kept in a gap with the conjugate pad without contact; after a period of liquid sample collection time; allowing the hydrogel to swell and connect with the conjugate pad; the pathogens or biochemical molecules bonded with the aptamer-modified gold nanoparticles in the flowing droplets reach the test/control color development detection area.

13. The device according to claim 11; wherein the screening test piece production program comprising of; on the carrier board
(a) immobilizing streptavidin on the nitrocellulose membrane substrate; on the test line and the control line;
(b) combining the biotinylated aptamer specific to the target pathogen with the streptavidin on the test line; at the same time; combine the complementary DNA fragment of the control aptamer with the streptavidin on the control line;
(c) arranging an absorbing layer at the right end of the nitrocellulose membrane substrate to guide the sample to effectively flow through the test line and the control line,
(d) immobilizing two kinds of aptamer-modified gold nanoparticles on the conjugate pad, and the aptamers combined with gold nanoparticles through thiolation, one of which is the aptamer-modif1ed gold nanoparticles for control, and the other for the aptamer-modified gold nanoparticles for detection, a liquid buffer segment is placed between the sample collection area and the conjugate pad, thus completing the screening test piece.

14. The deVice according to claim 11, the screening test piece production program comprising of, on the carrier substrate,
(a) immobilizing streptavidin on the nitrocellulose membrane substrate, on the test line and the control line,
(b) binding the aptamer for capture, that is, the biotinylated aptamer specific to the target pathogen, to the streptavidin on the test line,
(c) arranging an absorbing layer at the right end of the nitrocellulose membrane substrate to guide the sample to effectively flow through the test line and the control line,
(d) immobilizing the aptamer for detection on the conjugate pad, two options for the aptamer, one is the same as the aptamer for capture, and the other is different from the aptamer for capture, the aptamer is thiolated with gold nanoparticles (AuNPs) combined into the aptamer-modified gold nanoparticles,
(e) combining the complementary DNA fragment of the aptamer for detection with streptavidin on the control line, placing a liquid buffer fragment between the sample collection area and the conjugate pad, thus completing the screening test piece.

15. The device according to claim 11, wherein the screening test piece production program comprising of, on the carrier substrate
(a) immobilizing streptavidin/biotinylated aptamer on the test line on the nitrocellulose membrane substrate, and streptavidin on the control line,
(b) arranging an absorbing layer on the right end of the nitrocellulose membrane substrate to guide the sample to effectively flow through the test line and the control line,
(c) pre-soaking or dripping the sample collection layer into the extract, and after it is dried, a biotinylated aptamer modified gold nanoparticles (AuNPs) is immobilized on the conjugate pad, which is compatible with the extracted target analyte, and the highly specific aptamer is combined with gold nanoparticles (AuNPs) through thiolation, a hydrogel block or a liquid buffer segment is placed between the sample collection layer and the conjugate pad, thus completing the screening test piece.

16. The device according to claim 11, wherein the screening test piece production program comprising of, on the carrier substrate
(a) immobilizing streptavidin (streptavidin) on the nitrocellulose membrane substrate, and placing it on the control line,
(b) conjugating angiotensin-converting enzyme 2 (ACE2) specific for the target pathogen to 1H, 1H, 2H, 2H-perfluorododemayethiol (PFDT) in the test line, the body binds to streptavidin on the control line,
(c) arranging an absorbing layer at the right end of the nitrocellulose membrane substrate to guide the sample to effectively flow through the test line and the control line,
(d) immobilizing two aptamer-modif1ed gold nanoparticles on the conjugate pad, for the aptamer-modif1ed gold nanoparticles used for detecting the spike protein, a liquid buffer segment is placed between the sample collection layer and the detection area of the conjugate pad, thus completing the screening test piece.

17. The device according to claim 11, wherein the pathogen is selected from exhaled breath condensate (BBC) and exhaled aerosol (EBA), exhaled breath condensate includes semi-volatile, non-volatile, volatile organic compounds, cytokines, proteins, cellular debris, DNA and viruses, bacteria, exhaled aerosols contain microscopic aerosols produced by surface membrane disruption at alveolar level and upper airway turbulence.

18. The device according to claim 11, wherein the pathogen is selected from one or a combination of COVID-19 virus, influenza virus, Mycobacterium tuberculosis, Ebola virus, Zika virus, and norovirus.

19. The device according to claim 11, wherein the aptamers specific to the target pathogen are selected from one or a combination of Mycobacterium tuberculosis virulence factors (Fpr, FbpB and pr), Mycobacterium tuberculosis-specific proteins (phosphate-binding transport tuberculosis protein PstS1), M. tuberculosis extracellular antigens (MPT64 and MPT51), endothelial M. tuberculosis-specific proteins (ct-Crystalline, Acr and HspX) and soluble M. tuberculosis proteins (CFP-Z, -10, -30 and ESAT-6), surface lipoglycan (ManLAM).


SEQUENCE LISTING (8 sequences)

SEQ ID NO: 1 - DNA, 44 bp, synthetic construct
  source 1..44: mol_type = other DNA
gggaacaatatgttcaagggctctttaaagttttagttcgtttg

SEQ ID NO: 2 - DNA, 28 bp, synthetic construct
  source 1..28: mol_type = other DNA
agggctttttttttttttagttcgtttg

SEQ ID NO: 3 - DNA, 35 bp, synthetic construct
  source 1..35: mol_type = other DNA
tgggagctgatgtcgcatgggttttgatcacatga

SEQ ID NO: 4 - DNA, 34 bp, synthetic construct
  source 1..34: mol_type = other DNA
ttcgggaatgattatcaaatttatgccctctgat

SEQ ID NO: 5 - DNA, 30 bp, synthetic construct
  source 1..30: mol_type = other DNA
ggcgccatagcgacggggccattccaagaa

SEQ ID NO: 6 - DNA, 39 bp, synthetic construct
  source 1..39: mol_type = other DNA
ttggttgctgaatcccctcgtcttggcttctttgtcggg

SEQ ID NO: 7 - DNA, 58 bp, synthetic construct
  source 1..58: mol_type = other DNA
gctggatgtcgcttacgacaatattccttaggggcaccgctacattgacacatccagc

SEQ ID NO: 8 - DNA, 52 bp, synthetic construct
  source 1..52: mol_type = other DNA
gtacttccggaccagttgtctttcggtctctacccccagcccgtcaaaagtg